(19) United States
(12) Patent Application Publication
(10) Pub. No.: US 2020/0317738 A1
(43) Pub. Date: Oct. 8, 2020

(54) METHODS FOR INCREASING PROLIFERATION OF MAMMALIAN CELLS

(71) Applicant: University College Cork - National University of Ireland, Cork, Cork (IE)

(72) Inventors: Mary O'Connell Motherway, Co. Cork (IE); Douwe Van Sinderen, Co. Cork (IE); Fergus Shanahan, Co. Cork (IE); Aileen Houston, Co. Cork (IE)

(21) Appl. No.: 16/303,483

(22) PCT Filed: May 26, 2017
(86) PCT No.: PCT/EP2017/062798

§ 371 Date: Nov. 20, 2018

(30) Foreign Application Priority Data
May 26, 2016 (EP) 16171597.4

(51) Int. Cl. C07K 14/345; A23L 33/135; A61K 35/745

(52) U.S. Cl. CPC C07K 14/345; A61K 9/0053; A61K 35/745; A23L 33/135

(57) ABSTRACT

The invention describes peptide, and proteins and fusion proteins comprising the peptides, that are capable of increasing the proliferation of cells, especially epithelial cells in the mammalian gastrointestinal tract. The peptides comprise the hydrophilic domain of the TadE subunit of the Tad pilus of Bifidobacterium, especially the pilus encoded by the Tad2003 gene cluster identified in Bifidobacterium breve UCC2003, and functional fragments of the hydrophilic domain. The invention informs new treatments for diseases and conditions that involve attenuated epithelial cell proliferation and growth, such as inflammatory conditions of the gastrointestinal tract and trauma of the skin including burns and other wounds. The invention also informs treatments of newborn babies to promote proliferation and growth of gut epithelial tissue by administration of agents comprising the peptide of the invention in the form of infant formula.

### FIELD OF THE INVENTION

The present invention relates to proteins and peptides capable of causing an increase in proliferation of mammalian cells, especially mammalian epithelial cells, expression vectors encoding the proteins and peptides, and cells engineered for heterologous expression of DNA encoding the proteins and peptides. Also contemplated are methods of treating disease or conditions characterised by attenuated cellular growth, for example damaged epithelial tissue caused by inflammatory conditions of the mammalian gastrointestinal tract, and trauma of the skin.

### BACKGROUND TO THE INVENTION

Development of the human gut microbiota commences at birth, with bifidobacteria being among the first colonizers of the sterile newborn gastrointestinal tract. To date, the genetic basis of Bifidobacterium colonization and persistence remains poorly understood. Transcriptome analysis of the Bifidobacterium breve UCC2003 2.42-Mb genome in a murine colonization model revealed differential expression of a type IVb tight adherence (Tad) pilus-encoding gene cluster designated “tad₂₀₀₃.” Mutational analysis demonstrated that the tad₂₀₀₃ gene cluster is essential for efficient in vivo murine gut colonization, and immunogold transmission electron microscopy confirmed the presence of Tad pili at the poles of B. breve UCC2003 cells. Conservation of the Tad pilus-encoding locus among other B. breve strains and among sequenced Bifidobacterium genomes supports the notion of a ubiquitous pili-mediated host colonization and persistence mechanism for bifidobacteria.

### SUMMARY OF THE INVENTION

The Applicant has discovered a peptide, the hydrophilic domain of the TadE protein (Bbr_0137) of the Tad pilus from Bifidobacterium breve UCC2003, and fragments thereof, that have a significant proliferative effect of mammalian cells, especially mammalian epithelial cells. The invention has applications in the treatment of diseases or conditions characterised by damaged or dysregulated cellular growth, for example inflammatory conditions of the gut or skin conditions caused by disease or trauma, and in promoting the development of gut epithelial lining in infants. The Applicant has shown that Bifidobacterium breve UCC2003, a strain that expresses the Tad pilus, causes increased proliferation of epithelial cells in-vivo and that knock-down of the Tad pilus operon in the strain causes a loss of the proliferative effect. In addition, the Applicant has demonstrated that strains of bacteria that do not harbour the Tad pilus operon do not cause an increase in proliferation of epithelial cell when tested in-vitro, but that the same strains when engineered for heterologous expression of the Tad pilus cause significant proliferation of epithelial cells in-vitro. The Applicant has expressed and tested the protein subunits making up the Tad pilus and identified the hydrophilic domain of the TadE protein, and a short N-terminal fragment thereof, as being the active agent capable of effecting a significant increase in proliferation of epithelial cells.

The invention therefore relates to the TadE pilus subunit from the Tad pilus of Bifidobacterium breve UCC2003 and bioactive homologs of the TadE pilus subunit from different bacteria (for example different Gram positive bacteria, or different strain of Bifidobacterium), (hereafter “TadE protein” or “TadE protein of the invention”) or a hydrophilic domain thereof or functional fragments of the hydrophilic domain (hereafter “TadE peptide” or “TadE peptide of the invention”), in an isolated form.

In another aspect, invention provides a fusion protein comprising a TadE protein of the invention, or a TadE peptide of the invention, fused to a partner protein or peptide (hereafter “TadE fusion protein” or “TadE fusion protein of the invention).

The terms “TadE protein of the invention”, “TadE peptide of the invention”, and “TadE fusion protein of the invention” are hereafter collectively referred to as “TadE polyamino acid sequence” or “TadE polyamino acid sequence of the invention”. Examples of TadE polyamino acid sequences of the invention include TadE protein of B. breve UCC2003 (SEQUENCE ID NO:1), the hydrophilic domain of the TadE protein of B. breve UCC2003 (SEQUENCE ID NO:3), functional fragment of the hydrophilic domain (SEQUENCE ID NO: 30), functional homologs of the TadE protein of B. breve UCC2003 (FIG. 4), hydrophilic domains of the functional homologs, and functional fragments of the hydrophilic domains of the functional homologs of the TadE protein of B. breve UCC2003.

In another aspect, invention provides a nucleic acid encoding a TadE polyamino acid sequence of the invention. In one embodiment, the nucleic acid is a DNA molecule. In one embodiment, the nucleic acid is a cDNA. In one embodiment, the invention provides a cDNA encoding a TadE peptide of the invention. In one embodiment, the invention provides a cDNA encoding a TadE protein of the invention. In one embodiment, the invention provides a cDNA encoding a polyamino acid sequence comprising a sequence of SEQ ID NO: 3 or 30. In one embodiment, the invention provides a cDNA encoding a polyamino acid sequence comprising a sequence of SEQ ID NO: 1.

In another aspect, the invention provides an expression vector comprising DNA encoding a TadE polyamino acid sequence of the invention, in which the vector is configured for heterologous expression of the TadE polyamino acid sequence of the invention, in a host cell (hereafter “expression vector of the invention”). In one embodiment, the vector comprises DNA encoding the Tad pilus.

In another aspect, the invention provides a host cell, especially a bacterium or mammalian producer cell, engineered to heterologously express a TadE polyamino acid sequence of the invention (hereafter “transformed cell of the invention”). In one embodiment, the transformed host cell comprises an expression vector on the invention.

In another aspect, the invention provides a man-made composition comprising an active agent selected from: a TadE polyamino acid sequence of the invention; and a transformed host cell of the invention. In one embodiment, the composition is selected from a food, pharmaceutical or personal care compositions. In one embodiment, the food is in infant formula is powder or liquid form.

In another aspect, the invention provides a pharmaceutical composition comprising an active agent selected from: a TadE polyamino acid sequence of the invention; and a host cell that expresses a TadE polyamino acid sequence of the invention (hereafter “TadE active of the invention”), in combination with a suitable pharmaceutical excipient. In one embodiment, the pharmaceutical composition is formulated for oral delivery. In one embodiment, the pharmaceutical composition is formulated for gastric transit and release of the active agent is the gastrointestinal tract distal of the stomach, typically in the small or large intestine.

In one embodiment, the pharmaceutical composition is formulated for topical administration to the skin. The cell may be engineered for heterologous expression of TadE, engineered to overexpress a non-heterologous gene encoding a TadE polyamino acid sequence of the invention (for example the Tad pilus), or a wild-type TadE pilus expressing cell.

In another aspect, the invention provides a method for the treatment of a disease or condition in subject characterised by attenuated cellular growth of a target tissue, the method comprising the step of administration of a TadE active of the invention to the target tissue.

In another aspect, the invention provides methods for the treatment of an inflammatory disease of the gastrointestinal tract in a subject, the method comprising the step of administration of a TadE active of the invention to the epithelial tissue of the gastrointestinal tract.

In another aspect, the invention provides methods for increasing proliferation of cells, the method comprising the step of administration of a TadE active of the invention to the cells. In one embodiment, the method is carried out on cells in-vitro. In one embodiment, the method is carried out on cells in-vivo. In one embodiment, the method is carried out on cells ex-vivo. In one embodiment, the cells are epithelial cells. In one embodiment, the cells are neuronal cells. In one embodiment, the cells are vascular cells. In one embodiment, the cells are hepatic cells (hepatocytes, adipocytes or lipocytes). In one embodiment, the cells are kidney cells. In one embodiment, the cells are extracellular matrix cells. In one embodiment, the cells are contractile cells. In one embodiment, the cells are blood system cells. In one embodiment, the cells are immune system cells. In one embodiment, the cells are germ cells. In one embodiment, the cells are interstitial cells. In one embodiment, the cells are stem cells (including adult stem cells and embryonic stem cells. In one embodiment, the cells are progenitors of any one of the above cell types.

The invention also provides methods for promoting the growth of epithelial tissue in a subject, the method comprising the step of administration of a TadE active of the invention to the epithelial tissue. In one embodiment, the method is for promoting development of gut epithelial lining in infants. In one embodiment, the infant is a premature infant.

The invention also provides a method of producing a TadE polyamino acid sequence of the invention, comprising the steps of providing a transformed cell of the invention, culturing the transformed host cell to effect heterologous expression of recombinant polyamino acid sequence of the invention by the host cell, and recovering the recombinant polyamino acid sequence of the invention.

The invention also provides a method of engineering a cell for heterologous expression of a TadE polyamino acid sequence of the invention, comprising the steps of transforming the cell with an expression vector of the invention, whereby the transformed cell is capable of heterologous expression of a TadE polyamino acid sequence of the invention,

Other aspects and preferred embodiments of the invention are defined and described in the other claims set out below.

### BRIEF DESCRIPTION OF THE FIGURES

FIG. 1. Expression of B. breve TAD pili in L. lactis. (A) schematic representation of the tad₂₀₀₀₃ gene cluster expressed in L. lactis (B) Transmission electron micrograph of L. lactis cells expressing Tad₂₀₀₀₃ pili. (C) In-vitro epithelial proliferation assays performed with 3 epithelial cell lines, HT29, HCT116 or SW480.

FIG. 2. Analysis of in-vivo epithelial cell proliferation in colonic epithelial cells by immunostaining for Ki-67. Images represent the immunohistochemistry analysis of distal colonic tissue of C57black mice monoassociated with either B. breve UCC2003, B. breve UCC2003ΔtadE or B. breve UCC2003ΔtadF. Distal colonic tissue of control groups of Germ free or conventional C57black mice were included.

FIG. 3. Purification of the hydrophilic domain of TadE as a trxA fusion protein (A) Analysis of in-vitro epithelial proliferation with purified TrxA, TrxA-TadE or TrxA-TadF fusion proteins

FIG. 4. Tad locus gene alignment from different strains of Bifidobacterium showing TadE proteins having 39%-58% sequence identity with TadE from B. breve UCC2003.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

The invention relates to an isolated TadE polyamino acid sequence, for use as a medicament, or for use in a method of inducing proliferation of epithelial cells in a subject having epithelial cells damaged by disease, surgery, trauma, chemotherapy or radiotherapy, or for use in a method of development of gut epithelial lining in a premature infant, or for use in a method of treating or preventing a disease or condition in a subject characterised by attenuated cellular growth of a target tissue. The disease may be an inflammatory disease of the gastrointestinal tract.

The invention also relates to a pharmaceutical composition comprising a TadE polyamino acid sequence in combination with a suitable pharmaceutical excipient. The pharmaceutical composition may be formulated for oral delivery, and is optionally formulated for gastric transit and release of the polyamino acid sequence in the small or large intestine. The pharmaceutical composition may be formulated for topical administration to the skin.

The invention also provides an expression vector comprising DNA encoding a TadE polyamino acid sequence comprising SEQUENCE ID NO: 3, in which the vector is configured for heterologous expression of the polyamino acid sequence in a host cell. The expression vector is optionally selected from a bacterial plasmid, yeast plasmid, phage DNA, SV40, and a baculovirus.

The invention also provides a host cell engineered to heterologously express a TadE polyamino acid sequence. The invention also provides a host cell transformed with an expression vector of the invention. The host cell may be a bacterium, for example a non-pathogenic bacterium, a commensal bacterium, a probiotic bacterium, or a human gut bacterium.

The polyamino acid sequence may comprise SEQUENCE ID NO: 30. The polyamino acid sequence may comprise SEQUENCE ID NO: 3. The polyamino acid sequence may be a peptide having 8 to 70 amino acids and comprise SEQUENCE ID NO: 30. The polyamino acid sequence may be a peptide having up to 70 amino acids and comprise SEQUENCE ID NO: 3. The polyamino acid sequence may comprise SEQUENCE ID NO: 1.The polyamino acid sequence may consists essentially of SEQUENCE ID NO: 30. The polyamino acid sequence may be a fusion protein.

The invention also relates to a host cell of the invention, for use as a medicament, for use in a method of inducing proliferation of epithelial cells in a subject having epithelial cells damaged by disease, surgery, trauma, chemotherapy or radiotherapy, for use in a method of development of gut epithelial lining in a premature infant, in which the host cell is a optionally a bacterium, or for use in a method of treating or preventing a disease or condition in a subject characterised by attenuated cellular growth of a target tissue, and in which the disease is optionally an inflammatory disease of the gastrointestinal tract, and in which the host cell is optionally a bacterium.

In one embodiment, the inflammatory disease of the gastrointestinal tract is selected from mucocytis, colitis, Crohns disease, and inflammatory bowel disease.

The invention also provides an isolated TadE peptide. In one embodiment, the TadE peptide comprises SEQUENCE ID NO: 30 or SEQUENCE ID NO: 3.

The invention also provides an isolated nucleic acid encoding a TadE peptide. In one embodiment, the TadE peptide is a cDNA molecule.

The invention also provides a bacterium expressing a Tad pilus, for use in a method of inducing proliferation of epithelial cells in a subject having epithelial cells damaged by disease, surgery, trauma, chemotherapy or radiotherapy. In one embodiment, the epithelial cells are gut epithelial cells. The invention also provides a bacterium expressing a Tad pilus, for use in a method of development of gut epithelial lining in a premature infant. The invention also provides a bacterium expressing a Tad pilus, for use in a method of treating or preventing a disease or condition in a subject characterised by attenuated cellular growth of a target tissue. In one embodiment, the bacterium is engineered for heterologous expression of the Tad pilus. In one embodiment, the Tad pilus comprises SEQUENCE ID NO: 30 or SEQUENCE ID NO: 3. In one embodiment, the bacterium is a strain of Bifidobacterium breve. In one embodiment, the bacterium is Bifidobacterium breve UCC2003. In one embodiment, the bacterium is a probiotic bacterium, optionally selected from Streptococcus thermophilus, Bacillus laterosporus, Pediococcus acidilactici, Bifidobacterium infantis, Bifidobacterium bifidum, Bifidobacterium lactis, Bifidobacterium longum, Lactobacillus acidophilus, Lactobacillus brevis, Lactobacillus bulgaricus, Lactobacillus casei, Lactobacillus gasseri, Lactococcus lactis, Lactobacillus plantarum, Lactobacillus paracasei, Lactobacillus rhamnosus, Lactobacillus salivarius.

The invention also provides a food or dietary supplement composition comprising an isolated TadE polyamino acid sequence. In one embodiment, the isolated TadE polyamino acid sequence comprises SEQUENCE ID NO: 30, 3 or 1.

The invention also provides a food or dietary supplement composition comprising a bacterium according to the invention.

Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an “effective amount” or a “therapeutically effective amount” of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

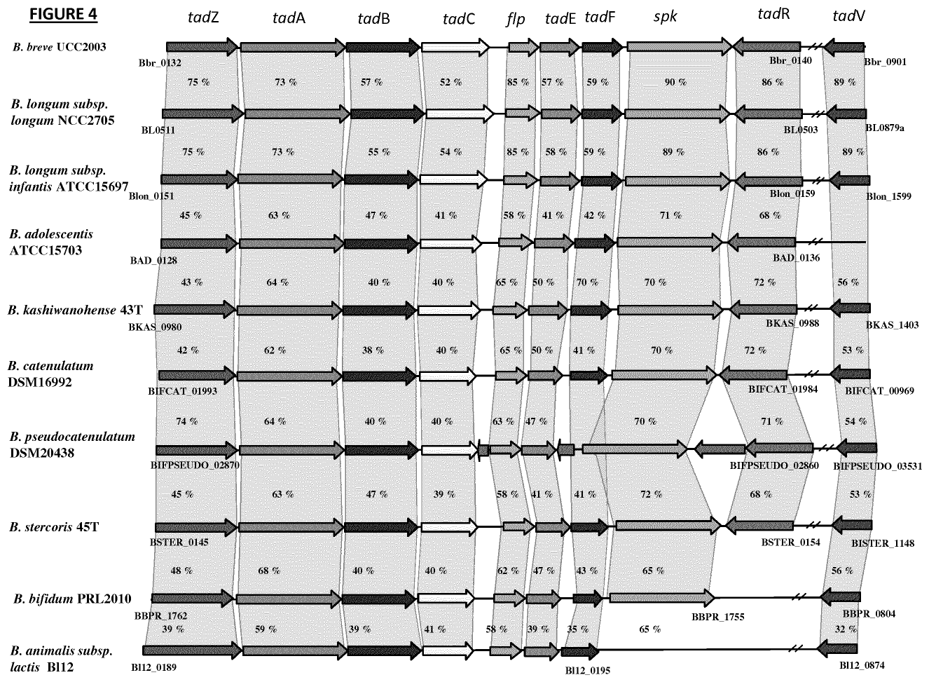

As used herein, the term “Tad pilus” refers to the type IVb tight adherence (Tad) pilus, a protein fibre appendage found on Gram positive bacteria including Bifidobacterium breve UCC2003. It is described in O'Connell Motherway et al (Proc Natl Acad Sci USA 108(27):11217-11222. Tad pili are also found on other strains of bacteria (FIG. 4).

As used herein, the term “TadE pilus subunit” refers to the TadE protein forming part of the Tad pilus appendage decorating the poles of Bifidobacterium breve UCC2003, and encoded by the Bbr_0137 gene of the Bifidobacterium breve UCC2003 genome. The amino acid and nucleic acid sequences of the TadE subunit of the Tad pilus of Bifidobacterium breve UCC2003 are provided in SEQUENCE ID NO's 1 and 2, respectively.

SEQUENCE ID NO: 1
MNMRKGLSRWLARLPPVHNEGAATAEFAVVLPSVIAITGVLLTLTRVVAV
SMDCQSAATASARELVVSDDESAAQQAAVNIVGSRVDVTVHREGSLVRVA
VECPVLPGPLNVTPTRVHGEAVAARQ
SEQUENCE ID NO: 2
atgaacatgagaaaaggtctcagcagatggctcgcccgtttgccgcctgt
gcacaatgagggtgcggccacggccgagttcgcggtagtgctgcccagcg
tcattgcaataacgggggtgttgctgacgcttacccgagtggttgcggta
tccatggattgccaaagtgccgcaacggcatccgcgcgtgaattggtggt
ttctgatgatgaatccgcagcacaacaggctgccgtgaatattgtgggaa
gccgagtggatgtgactgtgcatcgggaaggcagcttggtgcgagtggcg
gtcgaatgccccgttttgccagggcctttgaatgtgactcctactcgggt
gcatggtgaagctgtggctgcgcggcaataa

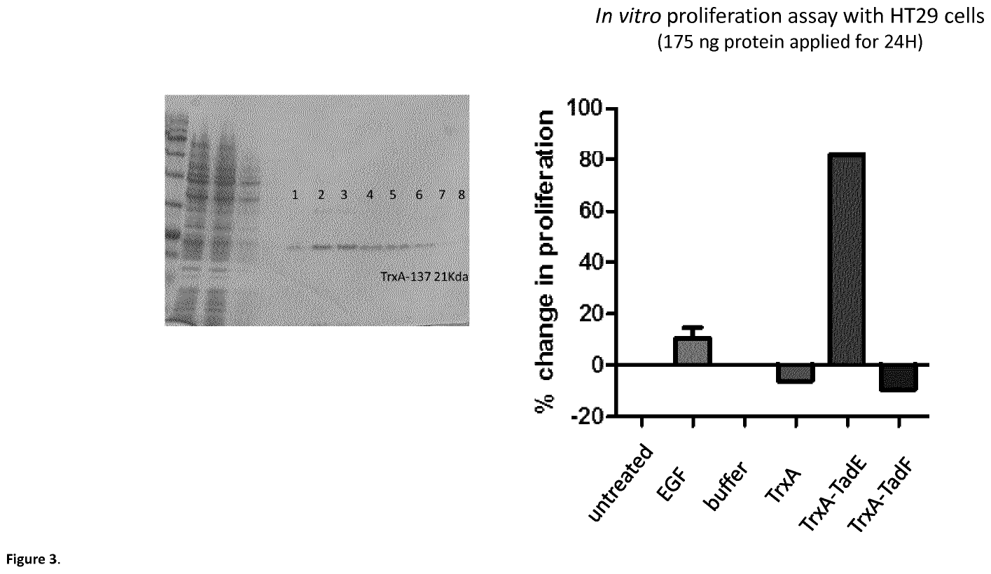

As used herein, the term “TadE protein” refers to the protein of SEQ ID NO: 1, and functional homologs thereof. Homologs of the TadE protein of SEQ ID NO: 1 can be found in different strains of bacteria that express a Tad pilus, for example different strains of Bifidobacterium breve including B. longum (for example B. longum subsp. longum NCC2705 and B. longum subsp. Infantis ATCC15697), B. adolescentis (for example B. adolescentis ATCC15703), B. kashiwanohense 43T, B. catenulatum DSM16992, B. pseudocatenulatum DSM20438, B. stercoris 45T, B. bifidum PRL2010, and B. animalis subsp. Lactis BI12. FIG. 4 shows a tad locus alignment of different Bifidobacterium strains, providing details of tadE genes having 39% to 58% sequence identity with the nucleic acid sequence of tadE from B. breve UCC2003 (SEQUENCE ID NO: 2). Thus, the term “functional homolog” includes proteins encoded by these genes. Typically, the functional homolog has at least 35% sequence identity with SEQUENCE ID NO: 1. The term “functional” as applied to homologs of the TadE protein of SEQ ID NO: 1 means homologs that are capable of causing an increase in proliferation of HT 29, HCT116 or SW480 cells in the in-vitro assay described below with reference to FIG. 3.

As used herein, the term “TadE peptide” refers to a polyamino acid sequence having up to 100, 90, 80, or 70 amino acids and comprising (a) the hydrophilic domain of the TadE protein from Bifidobacterium breve UCC2003 (or) the hydrophilic domain of a functional homologs of TadE protein (i.e see FIG. 4), and functional variants or fragments thereof. The amino acid and nucleic acid sequences of the active (hydrophilic) domain of the TadE subunit are provided in SEQUENCE ID NO's 3 and 4, respectively.

SEQUENCE ID NO: 3
SARELVVSDDESAAQQAAVNIVGSRVDVTVHREGSLVRVAVECPVLPGPL
NVTPTRVHGEAVAARQ-
SEQUENCE ID NO: 4
Tccgcgcgtgaattggtggtttctgatgatgaatccgcagcacaacaggc
tgccgtgaatattgtgggaagccgagtggatgtgactgtgcatcgggaag
gcagcttggtgcgagtggcggtcgaatgccccgttttgccagggcctttg
aatgtgactcctactcgggtgcatggtgaagctgtggctgcgcggcaata
a

Functional fragments of the hydrophilic domain of the TadE protein from Bifidobacterium breve UCC2003 are provided in SEQUENCE ID NO'S 30 to 35. Typically the fragments comprise SEQUENCE ID NO: 30.

(SEQUENCE ID NO: 30)
VHGEAVAARQ
(SEQUENCE ID NO: 31)
PLNVTPTRVHGEAVAARQ
(SEQUENCE ID NO: 32)
RVAVECPVLPGPLNVTPTRVHGEAVAARQ
(SEQUENCE ID NO: 33)
TRVHGEAVAAR
(SEQUENCE ID NO: 34)
TPTRVHGEAVAA

The active agents of the invention thus include TadE proteins of the invention including SEQUENCE ID NO. 1 and functional homologs thereof (i.e. TadE proteins encoded by tadE genes of FIG. 4), and functional variants thereof. The term functional variants as applied to the TadE proteins of the invention should be understood to mean variant proteins having substantially identical sequences (i.e. at least 90% sequence homology, or having from 1 to 10 amino acid changes including additions, deletions or substitutions) and that are capable of causing an increase in proliferation of HT29, HCT116 or SW480 cells in the in-vitro assay described below with reference to FIG. 3). The active agent of the invention also includes TadE peptides of the invention, including peptides comprising or consisting of the hydrophilic domain of B. breve UCC2003 TadE protein (SEQUENCE ID NO: 3) and peptides comprising or consisting of the hydrophilic domain of the TadE homologs of FIG. 4, and functional variants and fragments thereof. The term functional variants as applied to the TadE peptides of the invention should be understood to mean variant peptides having substantially identical sequences (i.e. at least 90% sequence homology, or having 1, 2, 3, 4, 5, 6, 7, 8, 9 or 10 amino acid changes including additions, deletions or substitutions) and that are capable of causing an increase in proliferation of HT29, HCT116 or SW480 cells in the in-vitro assay described below with reference to FIG. 3). The term functional fragment as applied to the TadE peptides of the invention should be understood to mean fragments of the peptides having about 8, 9 or 10 amino acids and that are capable of causing an increase in proliferation of HT29, HCT116 or SW480 cells in the in-vitro assay described below with reference to FIG. 3.

As used herein, the term “TadE polyamino acid sequence” refers to a TadE protein, TadE peptide, or a TadE fusion protein.

As used herein, the term “polyamino acid sequence comprising SEQUENCE ID NO: 3” means a protein, fusion protein, polypeptide, or peptide, comprising SEQUENCE ID NO: 3 or a functional variant thereof. As used herein, the term “polyamino acid sequence comprising SEQUENCE ID NO: 30” means a protein, fusion protein, polypeptide, or peptide, comprising SEQUENCE ID NO: 30 or a functional variant thereof. When necessary, any of the polyamino acid sequences described herein can be chemically modified, for example to increase their stability. A chemically modified peptide or a peptide analog includes any functional chemical equivalent of the peptide characterized by its increased stability and/or efficacy in vivo or in vitro in respect of the practice of the invention. The term peptide analog also refers to any amino acid derivative of a peptide as described herein. A peptide analog can be produced by procedures that include, but are not limited to, modifications to side chains, incorporation of unnatural amino acids and/or their derivatives during peptide synthesis and the use of cross-linkers and other methods that impose conformational constraint on the peptides or their analogs. Examples of side chain modifications include modification of amino groups, such as by reductive alkylation by reaction with an aldehyde followed by reduction with NaBH₄; amidation with methylacetimidate; acetylation with acetic anhydride; carbamylation of amino groups with cyanate; trinitrobenzylation of amino groups with 2, 4, 6, trinitrobenzene sulfonic acid (TNBS); alkylation of amino groups with succinic anhydride and tetrahydrophthalic anhydride; and pyridoxylation of lysine with pyridoxa-5′-phosphate followed by reduction with NABH₄. The guanidino group of arginine residues may be modified by the formation of heterocyclic condensation products with reagents such as 2,3-butanedione, phenylglyoxal and glyoxal. The carboxyl group may be modified by carbodiimide activation via o-acylisourea formation followed by subsequent derivatization, for example, to a corresponding amide. Sulfhydryl groups may be modified by methods, such as carboxymethylation with iodoacetic acid or iodoacetamide; performic acid oxidation to cysteic acid; formation of mixed disulphides with other thiol compounds; reaction with maleimide; maleic anhydride or other substituted maleimide; formation of mercurial derivatives using 4-chloromercuribenzoate, 4-chloromercuriphenylsulfonic acid, phenylmercury chloride, 2-chloromercuric-4-nitrophenol and other mercurials; carbamylation with cyanate at alkaline pH. Tryptophan residues may be modified by, for example, oxidation with N-bromosuccinimide or alkylation of the indole ring with 2-hydroxy-5-nitrobenzyl bromide or sulphonyl halides. Tryosine residues may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate. Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

Calculations of “homology” or “sequence identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

Amino acid and nucleotide sequence alignments and homology, similarity or identity, as defined herein are preferably prepared and determined using the algorithm BLAST 2 Sequences, using default parameters (Tatusova, T. A. et al, FEMS Microbiol Lett, 174: 187-188 (1999)). Alternatively, the BLAST algorithm (version 2.0) is employed for sequence alignment, with parameters set to default values. BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8.

As used herein, the term “isolated” as applied to a TadE protein or TadE peptide means a protein or peptide which is isolated from its natural environment. In the case of a TadE protein, this means that the protein is isolated from the other subunits of the Tad pilus and generally made by means of a technical process. In the case of the TadE peptide, this means that the peptide is separated from all or part of the hydrophilic domains of the TadE protein and generally made by means of a technical process.

As used herein, the term “TadE fusion protein” or “TadE fusion protein of the invention” refers to a fusion protein comprising a TadE protein or TadE peptide fused to a partner polyamino acid sequence typically by means of a linker sequence. Examples of TadE fusion proteins are described below. Methods for making fusion proteins will be well known to a person skilled in the art and generally involve synthesis of a fusion gene comprising DNA coding for the first part of the fusion protein but with the stop codon deleted appended to DNA coding for the second part of the fusion protein in frame through ligation or overlap extension PCR.

As used herein, the term “expression vector of the invention” may be any suitable vector, including chromosomal, non-chromosomal, and synthetic nucleic acid vectors (a nucleic acid sequence comprising a suitable set of expression control elements) suitable for expression of a TadE polyamino acid sequence of the invention in a cell. Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, the TadE polyamino acid sequence-encoding nucleic acid molecule is comprised in a naked DNA or RNA vector, including, for example, a linear expression element (as described in, for instance, Sykes and Johnston, Nat Biotech 12, 355-59 (1997)), a compacted nucleic acid vector (as described in for instance U.S. Pat. No. 6,077,835 and/or WO 00/70087), or a plasmid vector such as pBR322, pUC 19/18, or pUC 118/119. Such nucleic acid vectors and the usage thereof are well known in the art (see, for instance, U.S. Pat. Nos. 5,589,466 and 5,973,972). In one embodiment, the DNA comprises an expression control sequence.

In one embodiment, the vector is suitable for expression of a polyamino acid sequence of the invention in a bacterial cell. Examples of such vectors include expression vectors such as BlueScript (Stratagene), pIN vectors (Van Heeke & Schuster, 1989, J Biol Chem 264, 5503-5509), pET vectors (Novagen, Madison, Wis.) and the like. In one embodiment, the expression vector may also or alternatively be a vector suitable for expression in a yeast system. Any vector suitable for expression in a yeast system may be employed. Suitable vectors include, for example, vectors comprising constitutive or inducible promoters such as yeast alpha factor, alcohol oxidase and PGH (reviewed in: F. Ausubel et al., ed., 1987, Current Protocols in Molecular Biology, Greene Publishing and Wiley InterScience New York; and Grant et al., 1987, Methods in Enzymol 153, 516-544). In other embodiments, the expression vector is suitable for expression in baculovirus-infected insect cells. (Kost, T; and Condreay, J P, 1999, Current Opinion in Biotechnology 10 (5): 428-33.)

Expression control sequences are engineered to control and drive the transcription of genes of interest, and subsequent expression of proteins in various cell systems. Plasmids combine an expressible gene of interest with expression control sequences (i.e. expression cassettes) that comprise desirable elements such as, for example, promoters, enhancers, selectable markers, operators, etc. In an expression vector of the invention, TadE polyamino acid sequence-encoding nucleic acid molecules may comprise or be associated with any suitable promoter, enhancer, selectable marker, operator, repressor protein, polyA termination sequences and other expression-facilitating elements.

“Promoter” as used herein indicates a DNA sequence sufficient to direct transcription of a DNA sequence to which it is operably linked, i.e., linked in such a way as to permit transcription of the TadE polyamino acid sequence-encoding nucleotide sequence when the appropriate signals are present. The expression of a TadE polyamino acid sequence-encoding nucleotide sequence may be placed under control of any promoter or enhancer element known in the art. Examples of such elements include strong expression promoters (e.g., human CMV IE promoter/enhancer or CMV major IE (CMV-MIE) promoter, as well as RSV, SV40 late promoter, SL3-3, MMTV, ubiquitin (Ubi), ubiquitin C (UbC), and HIV LTR promoters). In some embodiments, the vector comprises a promoter selected from the group consisting of SV40, CMV, CMV-IE, CMV-MIE, RSV, SL3-3, MMTV, Ubi, UbC and HIV LTR.

Nucleic acid molecules of the invention may also be operably linked to an effective poly (A) termination sequence, an origin of replication for plasmid product in E. coli, an antibiotic resistance gene as selectable marker, and/or a convenient cloning site (e.g., a polylinker). Nucleic acids may also comprise a regulatable inducible promoter (inducible, repressable, developmentally regulated) as opposed to a constitutive promoter such as CMV IE (the skilled artisan will recognize that such terms are actually descriptors of a degree of gene expression under certain conditions).

Selectable markers are elements well-known in the art. Under the selective conditions, only cells that express the appropriate selectable marker can survive. Commonly, selectable marker genes express proteins, usually enzymes, that confer resistance to various antibiotics in cell culture. In other selective conditions, cells that express a fluorescent protein marker are made visible, and are thus selectable. Embodiments include beta-lactamase (bla) (beta-lactam antibiotic resistance or ampicillin resistance gene or ampR), bls (blasticidin resistance acetyl transferase gene), bsd (blasticidin-S deaminase resistance gene), bsr (blasticidin-S resistance gene), Sh ble (Zeocin® resistance gene), hygromycin phosphotransferase (hpt) (hygromycin resistance gene), tetM (tetracycline resistance gene or tetR), neomycin phosphotransferase II (npt) (neomycin resistance gene or neoR), kanR (kanamycin resistance gene), and pac (puromycin resistance gene).

In certain embodiments, the vector comprises one or more selectable marker genes selected from the group consisting of bla, bls, BSD, bsr, Sh ble, hpt, tetR, tetM, npt, kanR and pac. In other embodiments, the vector comprises one or more selectable marker genes encoding green fluorescent protein (GFP), enhanced green fluorescent protein (eGFP), cyano fluorescent protein (CFP), enhanced cyano fluorescent protein (eCFP), or yellow fluorescent protein (YFP).

For the purposes of this invention, gene expression in eukaryotic cells may be tightly regulated using a strong promoter that is controlled by an operator that is in turn regulated by a regulatory protein, which may be a recombinant “regulatory fusion protein” (RFP). The RFP consists essentially of a transcription blocking domain, and a ligand-binding domain that regulates its activity. Examples of such expression systems are described in US20090162901A1, which is herein incorporated by reference in its entirety.

As used herein “operator” indicates a DNA sequence that is introduced in or near a gene in such a way that the gene may be regulated by the binding of the RFP to the operator and, as a result, prevents or allow transcription of the gene of interest, i.e. a nucleotide encoding a polypeptide of the invention. A number of operators in prokaryotic cells and bacteriophage have been well characterized (Neidhardt, ed., Escherichia coli and Salmonella; Cellular and Molecular Biology 2d. Vol 2 ASM Press, Washington D.C. 1996). These include, but are not limited to, the operator region of the LexA gene of E. coli, which binds the LexA peptide, and the lactose and tryptophan operators, which bind the repressor proteins encoded by the Lad and trpR genes of E. coli. These also include the bacteriophage operators from the lambda PR and the phage P22 ant/mnt genes, which bind the repressor proteins encoded by lambda cl and P22 arc. In some embodiments, when the transcription blocking domain of the RFP is a restriction enzyme, such as NotI, the operator is the recognition sequence for that enzyme. One skilled in the art will recognize that the operator must be located adjacent to, or 3′ to the promoter such that it is capable of controlling transcription by the promoter. For example, U.S. Pat. No. 5,972,650, which is incorporated by reference herein, specifies that tetO sequences be within a specific distance from the TATA box. In specific embodiments, the operator is preferably placed immediately downstream of the promoter. In other embodiments, the operator is placed within 10 base pairs of the promoter.

In an exemplary cell expression system, cells are engineered to express the tetracycline repressor protein (TetR) and a protein of interest is placed under transcriptional control of a promoter whose activity is regulated by TetR. Two tandem TetR operators (tetO) are placed immediately downstream of a CMV-MIE promoter/enhancer in the vector. Transcription of the gene encoding the protein of interest directed by the CMV-MIE promoter in such vector may be blocked by TetR in the absence of tetracycline or some other suitable inducer (e.g. doxycycline). In the presence of an inducer, TetR protein is incapable of binding tetO, hence transcription then translation (expression) of the protein of interest occurs. (See, e.g., U.S. Pat. No. 7,435,553, which is herein incorporated by reference in its entirety.)

The vectors of the invention may also employ Cre-lox recombination tools to facilitate the integration of a gene of interest into a host genome. A Cre-lox strategy requires at least two components: 1) Cre recombinase, an enzyme that catalyzes recombination between two loxP sites; and 2) loxP sites (e.g. a specific 34-base pair by sequence consisting of an 8-bp core sequence, where recombination takes place, and two flanking 13-bp inverted repeats) or mutant lox sites. (See, e.g. Araki et al., 1995, PNAS 92:160-4; Nagy, A. et al., 2000, Genesis 26:99-109; Araki et al., 2002, Nuc Acids Res 30(19):e103; and US20100291626A1, all of which are herein incorporated by reference). In another recombination strategy, yeast-derived FLP recombinase may be utilized with the consensus sequence FRT (see also, e.g. Dymecki, S. M., 1996, PNAS 93(12): 6191-6196).

As used herein, the term “host cell” includes any cell that is suitable for expressing a recombinant nucleic acid sequence. Cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g. S. cerevisiae, S. pombe, P. partoris, P. methanolica, etc.), plant cells, insect cells (e.g. SF-9, SF-21, baculovirus-infected insect cells, Trichoplusia ni, etc.), non-human animal cells, mammalian cells, human cells, or cell fusions such as, for example, hybridomas or quadromas. In certain embodiments, the cell is a human, monkey, ape, hamster, rat or mouse cell. In other embodiments, the cell is eukaryotic and is selected from the following cells: CHO (e.g. CHO K1, DXB-11 CHO, Veggie-CHO), COS (e.g. COS-7), retinal cells, Vero, CV1, kidney (e.g. HEK293, 293 EBNA, MSR 293, MDCK, HaK, BHK21), HeLa, HepG2, WI38, MRC 5, Colo25, HB 8065, HL-60, Jurkat, Daudi, A431 (epidermal), CV-1, U937, 3T3, L cell, C127 cell, SP2/0, NS-0, MMT cell, tumor cell, and a cell line derived from an aforementioned cell. In some embodiments, the cell comprises one or more viral genes, e.g. a retinal cell that expresses a viral gene (e.g. a PER.C6® cell). In some embodiments, the cell is a CHO cell. In other embodiments, the cell is a CHO K1 cell. In one embodiment, the host cell is a bacterium, especially a probiotic bacterium. In one embodiment, the bacterium is selected from Bifidobacterium breve. In one embodiment, the bacterium is Bifidobacterium breve UCC2003. In one embodiment, the bacterium is a probiotic bacterium, optionally selected from Streptococcus thermophilus, Bacillus laterosporus, Pediococcus acidilactici, Bifidobacterium infantis, Bifidobacterium bifidum, Bifidobacterium lactis, Bifidobacterium longum, Lactobacillus acidophilus, Lactobacillus brevis, Lactobacillus bulgaricus, Lactobacillus casei, Lactobacillus gasseri, Lactococcus lactis, Lactobacillus plantarum, Lactobacillus paracasei, Lactobacillus rhamnosus, Lactobacillus salivarius.

As used herein, the term “transformed cell of the invention” refers to a host cell comprising a nucleic acid stably integrated into the cellular genome that comprises a nucleotide sequence coding for expression of a TadE polyamino acid sequence of the invention. In another embodiment, the present invention provides a cell comprising a non-integrated (i.e., episomal) nucleic acid, such as a plasmid, cosmid, phagemid, or linear expression element, which comprises a sequence coding for expression of a TadE polyamino acid sequence of the invention. In other embodiments, the present invention provides a cell line produced by stably transfecting a host cell with a plasmid comprising an expression vector of the invention.

As used herein, the term “engineered” as applied to a cell means genetically engineered using recombinant DNA technology, and generally involves the step of synthesis of a suitable expression vector (see above) and then transfecting the expression vector into a host cell (generally stable transfection).

As used herein, the term “heterologous expression” refers to expression of a nucleic acid in a host cell that does not naturally have the nucleic acid. Insertion of the nucleic acid into the heterologous host is performed by recombinant DNA technology.

As used herein, the term “TadE active of the invention” refers to a TadE polyamino acid of the invention, and a TadE expressing cell. The TadE expressing cell may be a cell that naturally contains a gene encoding a TadE protein (for example Bifidobacterium breve UCC2003), or it may be a cell that is genetically engineered for heterologous expression of a gene encoding a TadE protein, peptide or fusion protein (for example the engineered Lactococcus lactis strain described below which is engineered to express the B. breve UCC2003 Tad pilus encoding operon, including the TadE subunit).

As used herein, the term “disease or condition in a subject characterised by attenuated cellular growth of a target tissue” refers to diseases or conditions having a pathology that involves inhibition or growth of cells in a particular tissue. The cells may be epithelial cells, neuronal cells. In one embodiment, the cells are vascular cells. In one embodiment, the cells are hepatic cells (hepatocytes, adipocytes or lipocytes). In one embodiment, the cells are kidney cells. In one embodiment, the cells are extracellular matrix cells. In one embodiment, the cells are contractile cells. In one embodiment, the cells are blood system cells. In one embodiment, the cells are immune system cells. In one embodiment, the cells are germ cells. In one embodiment, the cells are interstitial cells. In one embodiment, the cells are stem cells (including adult stem cells and embryonic stem cells. In one embodiment, the cells are progenitors of any one of the above cell types. Examples include inflammatory disorders, especially those characterised by dysregulated expression of one or more cytokines which are downstream of NFκB such as TNF-α, IL-12 and IL-23 and/or over-activation of Toll-like Receptor 4 (TLR4), Toll-like Receptor 2 (TLR2) or Myeloid differentiating protein 88 (Myd88) adaptor-like protein (Mal). Inflammatory disorders may include skin inflammatory disorders, inflammatory disorders of the joints, inflammatory disorders of the cardiovascular system, certain autoimmune diseases, lung and airway inflammatory disorders, intestinal inflammatory disorders. Examples of skin inflammatory disorders include dermatitis, for example atopic dermatitis and contact dermatitis, acne vulgaris, and psoriasis. Examples of inflammatory disorders of the joints include rheumatoid arthritis. Examples of inflammatory disorders of the cardiovascular system are cardiovascular disease and atherosclerosis. Examples of autoimmune diseases include Type 1 diabetes, Graves disease, Guillain-barré disease, Lupus, Psoriatic arthritis, and Ulcerative colitis. Examples of lung and airway inflammatory disorders include asthma, cystic fibrosis, COPD, emphysema, and acute respiratory distress syndrome. Examples of intestinal inflammatory disorders include colitis and inflammatory bowel disease.

As used herein, the term “Inflammatory disease of the gastrointestinal tract” means an inflammatory disease of condition that involves inflammation of all or part of the digestive tract. Inflammatory bowel disease (IBD) is a collective term for a group of inflammatory conditions of the colon and small intestine—the primary diseases are Crohns disease and ulcerative colitis. In one embodiment, the inflammatory disease is an autoimmune disease.

Pharmaceutical compositions formulated or configured for oral administration and gastric transit are known in the art and include those described below:
Sathish et al (Int. J. Pharm. Sci. 2013; 258-269);
Kushal et al (Int. Res. J. Pharm. 2013, 4(3));
Philip et al (Oman Med J. 2010, 25(2));
Polymers for controlled drug delivery—Peter Tarcha (CRC Press, 21 Nov. 1990);
Pharmaceutical coating technology—Michael Aulton et al (Taylor & Francis 27 Oct. 1995);
http://www.slideshare.net/Balimusale/oral-controlled-drug-delivery-system;
European Patent No: 2418968 (Teagasc); and
European Patent No: 2097072 (RCSI).
Brayden et al. (European Journal of Pharmaceutical Sciences 79 (2015), 102-111.
Tambuwala et al. (Journal of Controlled Release 217 (2015) 221-227.
Zhang et al. Evaluation of alginate-whey protein microcapsules for intestinal delivery of lipophilic compounds in pigs (J. Sci. Food Agric. (2015).
Lamprecht et al. (Journal of Controlled Release 104 (2005) 337-346.
Hua et al. (Nanomedicine: Nanotechnology, Biology and Medicine 11 (2015) 1117-1132.
Drug Delivery: Fundamentals and Applications (Chapter 7, Oral Drug Delivery, Hillary and Brayden)

As used herein, the term “food” refers to a man-made food product including beverages, food additives and food supplements. Examples of foods include dairy products such as milk, yoghurt, cheese, cheese food, dairy powders, probiotic formulations, infant formula powders, follow-on milk formula, food for special medicinal purposes, meat products, soups, vegetable products, fruit juices, fruit products, breads, confectionary, cakes, sports supplements, nutritional supplements and the like.

As used herein, the term “personal care product” refers to products used by humans such as skin creams and lotions, scalp creams and lotions, aftershave lotion, shampoo and shower gels, conditioner, hair products such as hair gel, cream and lotions, toothpaste, dental floss and mouthwash products, wipes and tissues, and the like. In one embodiment, the personal care product is a product for topical application to the skin.

As used herein, the term “pharmaceutical composition” refers to a therapeutically effective amount of the therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. In the case of the present invention, the term “therapeutically effective amount” should be taken to mean an amount of therapeutic which results in a clinically significant increase in proliferation of target cells, for example gut epithelial cells or skin epithelial cells.

The term “carrier” refers to a diluent, adjuvant, excipient, or vehicle with which the Therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to, ease pain at the, site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al, Molecular Cloning: A Laboratory Manual, 2d ed. (1989) Cold Spring Harbor Laboratory Press, Cold Spring Harbor, N.Y. and Ausubel et al, Short Protocols in Molecular Biology (1999) 4th Ed, John Wiley & Sons, Inc. which are incorporated herein by reference) and chemical methods.

Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

Materials and Methods

Bacterial strains, and culture conditions. Bacterial strains used in this study are listed in Table 1. Bifidobacterium breve UCC2003 was routinely cultured in reinforced clostridial medium (RCM; Oxoid Ltd, Basingstoke, Hampshire, United Kingdom) or in de Man Rogosa and Sharpe Medium (MRS) (de Mann, Rogsa and Sharpe, 1960). Prior to inoculation the MRS was supplemented with cysteine-HCl (0.05%) Bifidobacterial cultures were incubated at 37° C. under anaerobic conditions which were maintained using an anaerobic hood (Davidson and Hardy, Belfast, Ireland). Escherichia coli was cultured in Luria Bertani broth (LB) (Sambrook et al., 1989) at 37° C. with agitation. Lactococcus lactis strains were cultivated in M17 broth containing 0.5% glucose (Terzaghi. and Sandine, 1975) at 30° C. Where appropriate growth media contained tetracycline (Tet; 10 μg ml⁻¹), chloramphenicol (Cm; 5 μg ml⁻¹ for E. coli or L. lactis, or 2.5 μg ml⁻¹ for B. breve), Spectinomycin (Spec; 100 μg ml⁻¹ for E. coli or B. breve) or kanamycin (Km; 50 μg ml⁻¹ for E. coli). Recombinant E. coli cells containing pBS424Δrep were selected on LB agar containing Spec.

Nucleotide sequence analysis. Sequence data were obtained from the Artemis-mediated (Rutherford et al., 2000) genome annotations of the B. breve UCC2003 sequencing project (O'Connell Motherway et al., 2011). Database searches were performed using non-redundant sequences accessible at the National Centre for Biotechnology Information internet site (http://www.ncbi.nlm.nih.gov) using Blast. Sequence alignments were performed using the Clustal Method of the MEGALIGN program of the DNASTAR software package (DNASTAR, Madison, Wis., USA).

DNA manipulations. Chromosomal DNA was isolated from B. breve UCC2003 as previously described (O'Riordan, 1998). Minipreparation of plasmid DNA from E. coli or L. lactis was achieved by using the Qiaprep spin plasmid miniprep kit (Qiagen GmBH, Hilden, Germany) as described previously (O'Connell Motherway et al., 2009). Single stranded oligonucleotide primers used in this study were synthesized by Eurofins (Ebersberg, Germany). Standard PCRs were performed using TaqPCR mastermix (Qiagen), while high fidelity PCR was achieved using KOD polymerase (Novagen, Darmstadt, Germany). B. breve colony PCRs were performed as described previously (O'Connell Motherway et al., 2009). PCR fragments were purified using the Qiagen PCR purification kit (Qiagen). Electroporation of plasmid DNA into E. coli was performed as described by Sambrook et al. (1989) and into L. lactis as described by Wells et al. (1993). Electrotransformation of B. breve UCC2003 was performed as described by O'Connell Motherway et al. (2009).

Plasmid Constructions. For the construction of plasmid pNZ-tadZ-spk, DNA fragments encompassing the tadZ (Bbr_0132) to tadC (Bbr_0135), or flp (Bbr_0136) to spk (Bbr_0139) genes were generated by PCR amplification from chromosomal DNA of B. breve UCC2003 using Q5 DNA polymerase and primer combinations tadZF and tadCR, and flpF and spkR, respectively. To facilitate translational fusion to the nisin promoter on pNZ8150 SmaI and XbaI or HindIII restriction sites were incorporated at the 5′ ends of each forward and reverse primer combination, respectively (Table 2). The two generated amplicons, TadZ-TadC or flp-spk, were digested with SmaI and XbaI or HindIII, and ligated into ScaI and XbaI or HindIII-digested nisin-inducible translational fusion plasmid pNZ8150 (Mierau and kleerebezem, 2005). The ligation mixtures were introduced into L. lactis NZ9000 (Table 1) by electrotransformation and transformants selected based on chloramphenicol resistance. The plasmid content of a number of Cm^{r} transformants was screened by restriction analysis and the integrity of positively identified clones was verified by sequencing. To create the final construct pNZ-tadZ-spk, the pNis promoter plus the flp-spk fragment were amplified from pNZ-flp-spk using pNisF and spkR. The XbaI and HindIII restriction sites incorporated at the 5′ ends of the forward and reverse primer combination facilitated cloning of the resultant fragment in the corresponding restriction sites of pNZ-tadZ-tadC. The plasmid content of a number of Cm^{r} transformants was screened by restriction analysis and the integrity of positively identified clones was verified by sequencing. The resulting construct was designated pNZ-tadZ-spk. For the construction of plasmid pPTPI-tadV a DNA fragment encompassing tadV (Bbr_0901) and it's predicted shine dalgarno sequence were generated by PCR amplification from chromosomal DNA of B. breve UCC2003 using Q5 DNA polymerase and primer combinations tadVF and tadVR. AfIIII and BamHI restriction endonuclease sequences incorporated in the forward and reverse primers respectively facilitated cloning in corresponding NcoI and BamHI sites of the nisin inducible transcriptional fusion vector pPTPI. The ligation mixture were introduced into E. coli EC101 (Table 1) by electrotransformation and transformants selected based on tetracycline resistance. The plasmid content of a number of tet^{r} transformants was screened by restriction analysis and the integrity of positively identified clones was verified by sequencing. One verified plasmid designated pPTPI-tadV was introduced by electroporation into L. lactis NZ9000 pNZ-tadZ-spk to generate L. lactis NZ9000 pNZ-tadZ-spk-pPTPI-tadV.

Cell lines and Proliferation assay with L. lactis cultures HT29, HCT116, SW480, colon epithelial cells (Rockville, Md.) were maintained in DMEM containing 10% FCS and penicillin-streptomycin. Cell proliferation was measured by resazurin reduction (Gonzalez and Tarloff, 2001). Cells were seeded at 2×10⁵ cells per milliliter in 96-well plates. After incubation with 10⁶ cfu of nisin induced L. lactis NZ9000 pNZ8150, pPTPI or L. lactis NZ9000 pNZ-tadZ-spk-pPTPI-tadV for 8 h, cells were washed and medium supplemented with 44 mM resazurin was added, and resazurin reduction to resorufin was measured fluorometrically using a GENios plate reader (Tecan, Grodig, Austria) and Xfluor spreadsheet software. Results obtained were expressed in fluorescence units (FU) and percentage viability was calculated as follows: (FU treated/FU control)×100. Values were normalized relative to the untreated cells.

Cloning, overproduction and purification of the hydrophilic domains of TadE and TadF For the construction of plasmids pNZtrxA-tadE and pNZtrxA-tadF DNA fragments encompassing the truncated tadE (from codon 61 to 127) and tadF genes (from codon 47 to codon 130) were generated by PCR amplification from chromosomal DNA of B. breve UCC2003 using Q5 polymerase and primer combinations TadEHF and TadEHR, and TadFHF and TadFHR, respectively. BamHI and XbaI restriction sites were incorporated at the 5′ ends of each forward and reverse primer combination, respectively (Table 2). The generated amplicons were digested with BamHI and XbaI, and ligated into similarly digested nisin-inducible thioredoxin fusion plasmid pNZ-trxA (Douillard et al., 2011). The ligation mixtures were introduced into L. lactis NZ9000 (Table 1) by electrotransformation and transformants selected based on chloramphenicol resistance. The plasmid content of a number of Cm^{r} transformants was screened by restriction analysis and the integrity of positively identified clones was verified by sequencing. For protein overproduction and purification 400 ml of M17 broth supplemented with 0.5% glucose was inoculated with a 2% inoculum of a particular L. lactis strain, followed by incubation at 30° C. until an Optical Density (O.D. at wavelength 600 nm) of 0.5 was reached, at which point protein overexpression was induced by the addition of the cell free supernatant of a nisin producing strain followed by continued incubation at 30° C. for 90 minutes. Cells were harvested by centrifugation, washed and concentrated 40-fold in lysis buffer (50 mM NaH₂PO₄, 300 mM NaCl, 10 mM imidazole; pH 8.0). Cell extracts were prepared using 106 μm glass beads and the mini-bead-beater-8 cell disrupter (Biospec Products, Bartville, Okla., USA). After homogenization the glass beads and cell debris were removed by centrifugation, while the supernatant containing the cytoplasmic fractions was retained. Protein purification from the cytoplasmic fraction was performed using Ni-NTA matrices in accordance with the manufacturers' instructions (Qiagen). Elution fractions were analysed by SDS polyacrylamide gel electrophoresis on a 12.5% polyacrylamide gel. After electrophoresis the gels were fixed and stained with Commassie Brilliant blue to identify fractions containing the purified protein. Rainbow prestained low molecular weight protein markers (New England Biolabs, Herdfordshire, UK) were used to estimate the molecular weight of the purified proteins.

Proliferation assay with purified TrxA-TadE or TrxA-TadF. HT29, colonic epithelial cells (Rockville, Md.) were maintained in DMEM containing 10% or 0.5% Foetal calf serum and penicillin-streptomycin. Cell proliferation was measured by resazurin reduction. Cells were seeded at 2×10⁵ cells per milliliter in 96-well plates and purified TrxA, TrxA-TadE or TrxA-TadF added at various concentrations. Following incubation for 24 h cells were washed and medium supplemented with 44 mM resazurin was added, and resazurin reduction to resorufin was measured fluorometrically as described above.

Construction of B. breve UCC2003ΔtadE or UCC2003ΔtadF deletion mutant strains. Isogenic non-polar deletion mutants of tadE (Bbr_0137), or tadF (Bbr_0138) with 276 bp of the 381 bp of tadE, or 327 bp of the 390 bp of tadF deleted were created using pBS423Δrep constructs generated by the splicing by overlap extension (SOEing) PCR procedure (Horton et al. 1990). In each case primers SOE AB and SOE CD (Table 2) were used to amplify regions flanking the sequence to be deleted using genomic DNA of B. breve UCC2003 as template. The resulting products, designated I or II were purified, mixed in a 1:1 ratio and used as template with primers SOE EF. The resulting product was digested with Pst1 and ligated to similarly digested pBS423Δrep (Hirayama et al., 2012) prior to transformation into E. coli EC101 by electroporation. Transformants were selected based on resistance to Kn and Spec and screened by colony PCR using primers pBSF and pBSR to identify clones harbouring the correct insert. The presence of the correct insert in a number of positive clones was confirmed by plasmid isolation and restriction analysis, while the sequence integrity of the cloned DNA fragment and the orientation of the insert in the pBS423Δrep vector was confirmed by sequencing. First crossover insertion mutations were generated essentially as described previously (O'Connell Motherway et al., 2009) to produce B. breve UCC2003 derivatives that were designated UCC2003-tadE-(I) or UCC2003-tadE-(II), or UCC2003-tadF-(I) or UCC2003-tadF-(II), respectively where I or II indicate that the first crossover occurred via fragment I or II (described above). Site-specific recombination in potential spec-resistant mutant isolates was confirmed by colony PCR using primer combinations specFw and specRv to verify spectinomycin gene integration, and primers tadESOE A or tadF2SOE A (positioned upstream of the selected flanking regions of tadE or tadF respectively), each in combination with pBSR or to confirm integration at the correct chromosomal location. To promote pBS423Δrep plasmid excision in UCC2003-tadE-(I) or UCC2003-tadE-(II), or UCC2003-tadF-(I) or UCC2003-tadF-(II) the incompatible plasmid pRTB101-CM was introduced into each strain and transformants were selected on RCA supplemented with CM. CM resistant colonies were subcultured for eight transfers to promote loss of integrated pBS423Δrep. Cells which had excised pBS423Δrep and had either reverted to the wild type genotype, or harboured a tadE or tadF deletion, were selected based on Cm^{r} Spec^{s}. Screening of Spec^{s} colonies for UCC2003 derivatives harbouring tadE or tadF deletion was performed by colony PCR using primer pairs tadESOEA and tadESOEB or tadF2SOEA and tadF2SOEB, respectively and sequencing of the PCR products to confirm the inframe deletion. Curing of pRTB101-CM from B. breve deletion mutant strains was performed by subculturing at 42° C. for 8 transfers followed by plating on RCA and screening for CM sensitive mutant strains by replica plating.

Colonization of Germ Free Mice Eight-week-old female, germ-free C57black mice were housed in flexible film gnotobiotic isolators under a strict 12 h light cycle. Mice were fed an autoclaved standard polysaccharide-rich mouse chow diet. Mice (n=7 per group) were inoculated by with 1×10⁹ cfu of B. breve UCC2003PK1, UCC2003ΔtadE or UCC2003ΔtadF or in 20 μl of PBS by oral pipetting whereby the inoculums are delivered by positioning a micropipette tip immediately behind the incisors. Five mice were maintained as uninoculated controls to monitor the germ free status of the facility. Fecal pellets were collected twice weekly to determine the number of each strain present. At day 30 the animals were sacrificed and their intestinal tracts quickly dissected. Aliquots of the small intestine, caecum and large intestine were retained in PBS for cfu determination (serial dilution plating on RCA agar supplemented with the appropriate antibiotics) while samples of the small intestine, caecum, proximal and distal colon were retained for immunohistochemistry analysis.

Immunohistochemistry analysis Immunohistochemical staining was performed on formalin-fixed, paraffin-embedded (FFPE) sections as previously described (Fernandes et al, 2015). Ki-67 Rabbit Monoclonal Antibody (Thermo Scientific) and Polyclonal Goat Anti-Rabbit Immunoglobulins/Biotinylated from Dako were used according to the manufacturer's protocol to analyze the cell cycle status. To quantify anti-Ki-67 positive cells, the ratio of anti-Ki-67 positive cells to the total cell count in the crypt was determined.

Results

Heterologous Expression of Tad₂₀₀₃ pili in L. lactis and Analysis of In-Vitro Proliferation

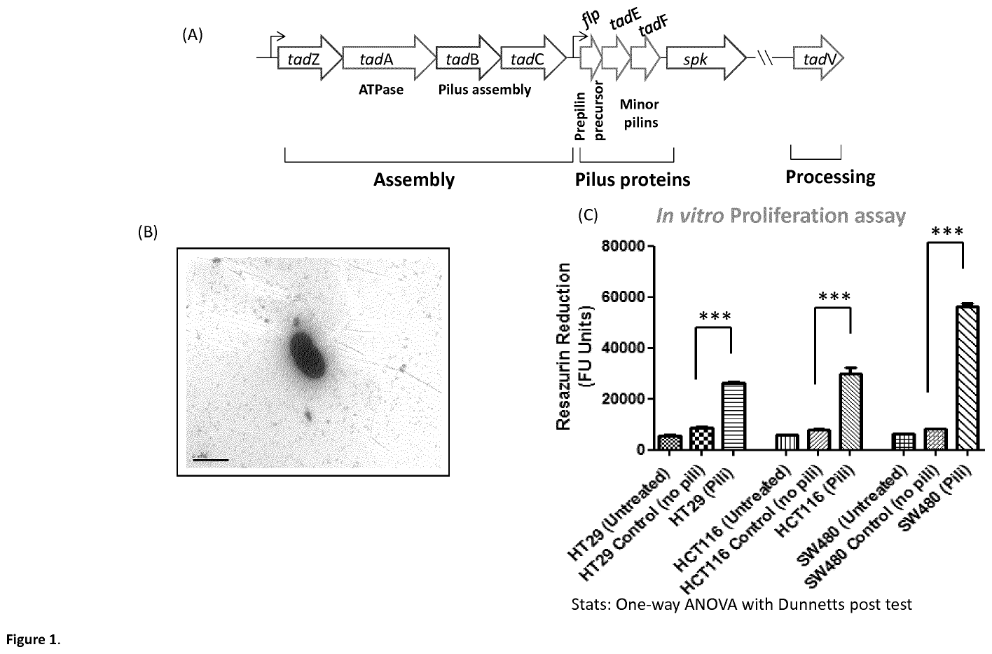

In order to establish if the B. breve UCC2003 Tad pili are solely responsible for the in vivo proliferation observed for this strain, the tad₂₀₀₃ pili cluster was cloned and expressed in L. lactis under the control of nisin inducible promoters. The resultant strain designated L. lactis NZ9000 pNZ-tadZ-spk-pPTPI-tadV and the corresponding control strain L. lactis NZ9000 pNZ8250-pPTPI (harbouring empty plasmids) were induced with nisin and incorporated in an in-vitro proliferation assay with three different colonic epithelial cell lines, namely HT29, HCT116 or SW480. Cell proliferation based on the ability of a viable, metabolically active cell, to reduce resazurin to resorufin and dihydroresorufin was measure fluorometrically. This analysis demonstrated that L. lactis producing Tad₂₀₀₃ pili significantly increased proliferation of the colonic epithelial cell lines as compared to the proliferation observed for cells incubated with control strain L. lactis NZ9000 pNZ8250-pPTPI (FIG. 1).

The B. breve UCC2003 tadE Encoded Pseudopilin Contributes to Tad₂₀₀₃ Mediated Epithelial Proliferation Under In Vivo Conditions

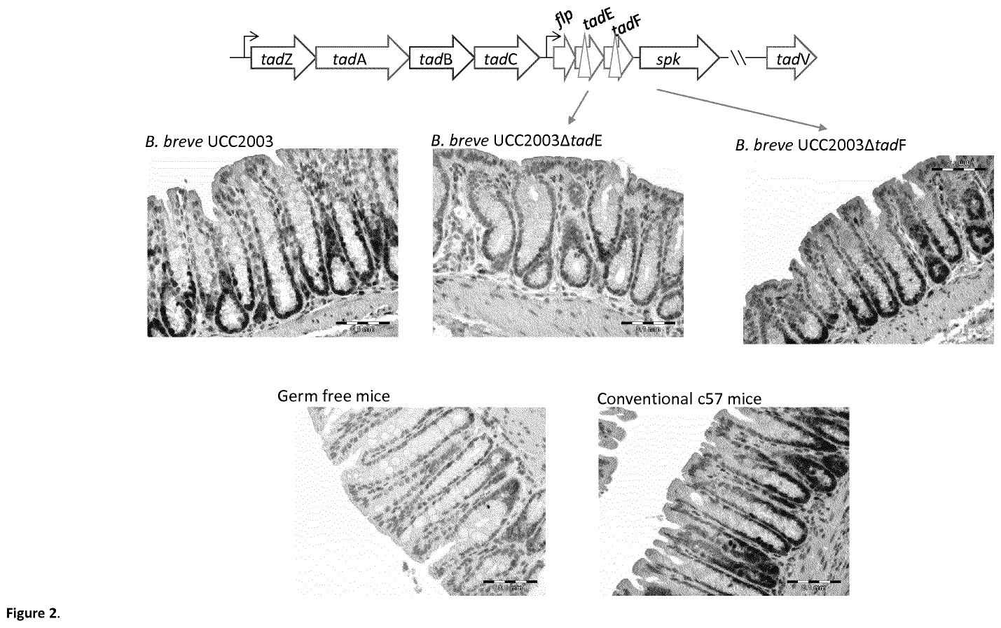

In order to demonstrate if either of the pseudopilins, TadE or TadF, contribute to the Tad₂₀₀₃ mediated epithelial proliferation phenotype, B. breve isogenic mutant strains, B. breve UCC2003ΔtadE or UCC2003ΔtadF, that harbour in-frame deletions of the tadE or tadF genes were constructed (see materials and methods). To investigate their relative colonization ability in germ-free mice, 1×10⁹ of B. breve UCC2003PK1 or B. breve UCC2003ΔtadE or UCC2003ΔtadF were administered by oral inoculation to eight week old germ-free C57 black mice (n=7) for three days. Fecal samples were collected twice weekly for the following four weeks and bacterial numbers were enumerated. The results demonstrated that each strain colonised the germ free murine gut at a level of approximately 10⁹ cfu g⁻¹ feces. At day 30 the mice were sacrificed, enumeration of Bifidobacteria in the intestine also reflected the high colonisation level of each strain. Immunohistochemistry analysis by Ki-67 staining for epithelial proliferation demonstrated proliferation in tissues recovered from mice administered B. breve UCC2003PK1, B. breve UCC2003ΔtadF and in the conventional C57Black mouse controls. Intriguingly an absence of proliferation in the distal colonic epithelia of mice that had been monoassociated with B. breve UCC2003ΔtadE was observed, and the distal colonic tissue was similar to tissue of the Germ free controls. This observation implicates the TadE pseudopilin as contributing to the in vivo Tad₂₀₀₃ mediated epithelial proliferation phenotype (FIG. 2).

Purified TadE Elicits Epithelial Proliferation Under In Vitro Conditions

In order to establish if heterologously expressed and purified TadE or TadF could elicit epithelial proliferation DNA fragments encoding the hydrophilic domains of tadE or tadF were cloned in the thioredoxin fusion plasmid pNZ-trxA, overexpressed and purified from L. lactis NZ9000. The purified fusion proteins TrxA-TadE, TrxA-TadF and control TrxA were incorporated in in vitro HT29 epithelial proliferation assays. The results demonstrate that that in contrast to TrxA or TrxA-TadF, the TrxA-TadE fusion protein elicits significant epithelial proliferation (FIG. 3). A fragment of the hydrophilic domain of TadE was also shown to elicit epithelial proliferation in-vitro.

TABLE 1
Bacterial strains

| Strains and plasmids | Relevant features | Reference or source |
|---|---|---|
| Strains | | |
| Escherichia coli strains | | |
| E. coli EC101 | Cloning host, repA⁺ km^{r} | (Law etal., 1995) |
| E. coli EC101-pNZ-M.BbrII + M.BbrIII | EC101 harbouring pNZ8048 derivative containing bbrIIM and bbrIIIM. | (O'Connell-Motherway et al., 2009) |
| E. coli EC101pBS423Δrep | EC101 harbouring pBS423Δrep | |
| E. coli EC101pBS423TadE1 + 2 | EC101 harbouring pBS423Δrep + tadE soeing fragments 1 + 2 | This study |
| E. coli EC101pBS423TadF1 + 2 | EC101 harbouring pBS423Δrep + tadF soeing fragments 1 + 2 | This study |
| Lactococcus lactis strains | | |
| L. lactis NZ9000 | MG1363, pepN::nisRK, nisin inducible overexpression host | (Mierau and kleerebezem, 2005) |
| L. lactis NZ9000 pNZ-tadZ-tadC | NZ9000 containing pNZ-tadZ-tadC | This study |
| L. lactis NZ9000- pNZ-flp-spk | NZ9000 containing pNZ-flp-spk | This study |
| L. lactis NZ9000- pNZ-tadZ-spk | NZ9000 containing pNZ-tadZ-spk | This study |
| L. lactis NZ9000- pPTPI-tadV | NZ9000 containing pPTPI-tadV | This study |
| L. lactis NZ9000 pNZtrxA-tadE | NZ9000 containing pNZtrxA-tadE | This study |
| L. lactis NZ9000 pNZtrxA-tadF | NZ9000 containing pNZtrxA-tadF | This study |
| L. lactis NZ9000 pNZ8150 pPTPI | NZ9000 containing pNZ8150 pPTPI | This study |
| Bifidobacterium sp. strains | | |
| B. breve UCC2003 | Isolate from nursling stool | ((O'Connell-Motherway et al., 2011)) |
| B. breve UCC2003ΔtadE | pORI19-tet-galA-967 insertion mutant of UCC2003 | This study |
| B. breve UCC2003ΔtadF | pORI19-tet-galG-410 insertion mutant of UCC2003 | This study |

TABLE 2
Oligonucleotide primers

| Purpose | Primer | Sequence^{a} |
|---|---|---|
| Cloning of tad operon | tadZF | CTGGACCCCGGGATGAATATCGACACATCCTCTCGCAC (SEQ ID 5) |
| in pNZ8150 | tadCR | TGCGCATCTAGACTACATCAGGAAAGAAGCAATTGC (SEQ ID 6) |
| | flpF | CTGGACCCCGGGATGGACACGCACATTCCCATCATC (SEQ ID 7) |
| | spkR | TGCGCAAAGCTTCTAGTTAAAGCGTCCGGTCAC (SEQ ID 8) |
| | pNisF | CTGGACTCTAGACTGACAATAGAAACATTAAC (SEQ ID 9) |
| Cloning of tadV in pPTPI | tadVF | AATTAAACATGTGGTGCAGGCGGGTG (SEQ ID 10) |
| | tadVR | CATCACGGATCCCAAGACAACCCCAACAAC (SEQ ID 11) |
| Cloning of tadE in | TadEHF | GACAAGGGATCCTCCGCGCGTGAATTGGTG (SEQ ID 12) |
| pNZ-trxA | TadEHR | AGCTCTCTAGATTATTGCCGCGCAGCCACAG (SEQ ID 13) |
| Cloning of tadF in | TadFHF | GACAAGGGATCCGCACGTTCCCTAGCTGACCT (SEQ ID 14) |
| pNZ-trxA | TadFHR | AGCTCTCTAGATTACTCACTAGCACCTCCC (SEQ ID 15) |
| Construction of B.breve | TadE soeA | GCAATTGCTTCTTTCCTGATGTAG (SEQ ID 16) |
| UCC2003ΔtadE | TadE soeB | GAGTAGGAGTCACATTCAAAGGAGGCGGCAAACGGGCGAGC (SEQ ID 17) |
| | TadE soeC | GCTCGCCCGTTTGCCGCCTCCTTTGAATGTGACTCCTACTC (SEQ ID 18) |
| | TadE soeD | CGTACCGGTAATGTCGATGAC (SEQ ID 19) |
| | TadE soeE | CCTGCACTGCAGGCACGGAGTCAGGAGAGCGCACG (SEQ ID 20) |
| | TadE soeF | GCTAGACTGCAGCTCCCTTATCGCATTCCGCCG (SEQ ID 21) |
| Construction of B.breve | TadF soeA | CTGAGTACGCGGTGGTGC (SEQ ID 22) |
| UCC2003ΔtadF | TadF soeB | CTTATCGCATTCCGCCGGTGACATACGTGGCATACA (SEQ ID 23) |
| | TadF soeC | TGTATGCCACGTATGTCACCGGCGGAATGCGATAAG (SEQ ID 24) |
| | TadF soeD | CTAGTTAAAGCGTCCGGTCAC (SEQ ID 25) |
| | TadF soeE | GCTCTACTGCAGGATGCCATTAAGACGTTGCTGAC (SEQ ID 26) |
| | TadF soeF | CTTGACCTGCAGGAAGTCACACGG (SEQ ID 27) |
| pBS423Δrep multiple cloning site primers | pBSF | 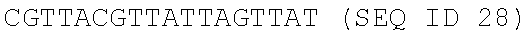 |
| | pBSF | GTAATACGTTCGTGTCGCG (SEQ ID 29) |

^{a}Restriction sites incorporated into oligonucleotide primer sequences are indicated in bold

### REFERENCES

1. De Man, J. C., A. Rogosa & M. E. Sharpe, (1960) A medium for the cultivation of lactobacilli. J Appl Bacteriol. 23: 130-135.
2. de Ruyter, P. G., O. P. Kuipers & W. M. de Vos, (1996) Controlled gene expression systems for Lactococcus lactis with the food-grade inducer nisin. Appl. Environ. Microbiol 62: 3662-3667.
3. Douillard F P, O'Connell-Motherway M, Cambillau C, van Sinderen D. (2011) Expanding the molecular toolbox for Lactococcus lactis: construction of an inducible thioredoxin gene fusion expression system. Microb Cell Fact. 2011 Aug. 9; 10:66. doi: 10.1186/1475-2859-10-66.
4. Gonzalez R J, Tarloff J B. Evaluation of hepatic subcellular fractions for Alamar blue and MTT reductase activity. (2001)Toxicol In Vitro. Jun; 15(3):257-9.
5. Hirayama Y, Sakanaka M, Fukuma H, Murayama H, Kano Y, Fukiya S, Yokota A (2012). Development of a double-crossover markerless gene deletion system in Bifidobacterium longum: functional analysis of the α-galactosidase gene for raffinose assimilation. Appl Environ Microbiol. Jul; 78(14):4984-94. doi: 10.1128/AEM.00588-12. Epub 2012 May 11.
6. Law J1, Buist G, Haandrikman A, Kok J, Venema G, Leenhouts K. (1995) A system to generate chromosomal mutations in Lactococcus lactis which allows fast analysis of targeted genes. J Bacteriol. 177(24):7011-8.
7. Mierau, I., and Kleerebezem, M. (2005) 10 years of the nisin-controlled gene expression system (NICE) in Lactococcus lactis. Appl Microbiol Biotechnol. 68:705-17
8. O'Connell-Motherway, M., J. O'Driscoll, G. F. Fitzgerald & D. van Sinderen, (2008) Overcoming the restriction barrier to plasmid transformation and targeted mutagenesis in Bifidobacterium breve UCC2003. Microbial Biotechnology 2: 321-332.
9. O'Connell Motherway M, Zomer A, Leahy S C, Reunanen J, Bottacini F, Claesson M J, O'Brien F, Flynn K, Casey P G, Moreno Munoz J A, Kearney B, Houston A M, O'Mahony C, Higgins D G, Shanahan F, Palva A, de Vos W M, Fitzgerald G F, Ventura M, O'Toole P W, van Sinderen D. 2011. Functional genome analysis of Bifidobacterium breve UCC2003 reveals type IVb tight adherence (Tad) pili as an essential and conserved host-colonization factor. Proceedings of the National Academy of Sciences 108:11217-11222.
10. O'Riordan, K. (1998). Studies on antimicrobial activity and genetic diversity of Bifidobacterium species: molecular characterization of a 5.75 kb plasmid and a chromosomally encoded recA gene homologue from Bifidobacterium breve. Ph.D. National University of Ireland, Cork, Cork.
11. Rutherford, K., Parkhill, J., Crook, J., Horsnell, T., Rice, P., Rajandream, M. A., and Barrell, B. (2000). Artemis: sequence visualization and annotation. Bioinformatics 10:944-945.
12. Sambrook, J., Fritsch, E. F., and Maniatis, T. (1989). Molecular cloning a laboratory manual, 2nd ed. Cold Spring Harbor Laboratory, Cold Spring Harbor, N.Y.
13. Terzaghi, B. E. and Sandine, W. E. 1975. Improved Medium for Lactic Streptococci and their Bacteriophages. Appl Microbiol. 29:807-813
14. Wells, J. M., Wilson, P. W. and Le Page, R. W. (1993) Improved cloning vectors and transformation procedure for Lactococcus lactis. J. Appl. Bacteriol. 74:629-36.

Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. An isolated TadE polyamino acid sequence, for use as a medicament.

2. An isolated TadE polyamino acid sequence, for use in a method of inducing proliferation of epithelial cells in a subject having epithelial cells damaged by disease, surgery, trauma, chemotherapy or radiotherapy.

3. An isolated TadE polyamino acid sequence, for use in a method of development of gut epithelial lining in a premature infant.

4. An isolated TadE polyamino acid sequence, for use in a method of treating or preventing a disease or condition in a subject characterised by attenuated cellular growth of a target tissue.

5. An isolated TadE polyamino acid sequence, for use of claim 4, in which the disease is an inflammatory disease of the gastrointestinal tract.

6. An isolated TadE polyamino acid sequence, for use of any of claims 1 to 5, in which the polyamino acid sequence comprises SEQUENCE ID NO: 30.

7. An isolated TadE polyamino acid sequence, for use of any of claims 1 to 5, in which the polyamino acid sequence comprises SEQUENCE ID NO: 3.

8. An isolated TadE polyamino acid sequence, for use of any of claims 1 to 5, in which the polyamino acid sequence is a peptide having 8 to 70 amino acids and comprising SEQUENCE ID NO: 30.

9. An isolated TadE polyamino acid sequence, for use of any of claims 1 to 5, in which the polyamino acid sequence is a peptide having up to 70 amino acids and comprising SEQUENCE ID NO: 3.

10. An isolated TadE polyamino acid sequence, for use of any of claims 1 to 5, in which the polyamino acid sequence comprises SEQUENCE ID NO: 1.

11. An isolated TadE polyamino acid sequence, for use of any of claims 1 to 5, in which the polyamino acid sequence consists essentially of SEQUENCE ID NO: 30.

12. An isolated TadE polyamino acid sequence, for use of any of claims 1 to 5, in which the polyamino acid sequence is a fusion protein.

13. A pharmaceutical composition comprising a TadE polyamino acid sequence in combination with a suitable pharmaceutical excipient.

14. A pharmaceutical composition as claimed in claim 13 formulated for oral delivery.

15. A pharmaceutical composition as claimed in claim 13 formulated for oral delivery and gastric transit and release of the polyamino acid sequence in the small or large intestine.

16. A pharmaceutical composition as claimed in claim 13 formulated for topical administration to the skin.

17. A pharmaceutical composition as claimed in any of claims 13 to 16, in which the polyamino acid sequence comprises SEQUENCE ID NO: 30.

18. A pharmaceutical composition as claimed in any of claims 13 to 16, in which the polyamino acid sequence comprises SEQUENCE ID NO: 3.

19. A pharmaceutical composition as claimed in any of claims 13 to 18, in which the polyamino acid sequence is a peptide having 8 to 70 amino acids.

20. A pharmaceutical composition as claimed in any of claims 13 to 19, in which the polyamino acid sequence is a fusion protein.

21. An expression vector comprising DNA encoding a TadE polyamino acid sequence comprising SEQUENCE ID NO: 3, in which the vector is configured for heterologous expression of the polyamino acid sequence in a host cell.

22. An expression vector as claimed in claim 21, in which the polyamino acid sequence comprises SEQUENCE ID NO: 30.

23. An expression vector as claimed in claim 21, in which the polyamino acid sequence comprises SEQUENCE ID NO: 3.

24. An expression vector as claimed in any of claims 21 to 23, in which the polyamino acid sequence is a peptide having 8 to 70 amino acids.

25. An expression vector as claimed in any of claims 21 to 24, in which the polyamino acid sequence is a fusion protein.

26. An expression vector as claimed in any of claims 21 to 25 selected from a bacterial plasmid, yeast plasmid, phage DNA, SV40, and a baculovirus.

27. A host cell engineered to heterologously express a TadE polyamino acid sequence.

28. A host cell transformed with an expression vector of any of claims 21 to 25.

29. A host cell transformed with an expression vector of any of claims 22 to 25.

30. A host cell according to claim 27, 28 or 29, which is a bacterium.

31. A host cell according to claim 29, which is a bacterium.

32. A host cell according to claim 31, which is a bacterium, and in which the bacterium is a human gut bacterium.

33. A host cell according to any of claims 27 to 32, for use as a medicament.

34. A host cell according to any of claims 27 to 32, for use in a method of inducing proliferation of epithelial cells in a subject having epithelial cells damaged by disease, surgery, trauma, chemotherapy or radiotherapy.

35. A host cell according to any of claims 27 to 32, for use in a method of development of gut epithelial lining in a premature infant, in which the host cell is a bacterium.

36. A host cell according to any of claims 27 to 32, for use in a method of treating or preventing a disease or condition in a subject characterised by attenuated cellular growth of a target tissue.

37. A host cell according to any of claims 127 to 32, for use of claim 28, in which the disease is an inflammatory disease of the gastrointestinal tract, and in which the host cell is a bacterium.

38. A host cell according to any of claims 27 to 32, for use of claim 37, in which the inflammatory disease of the gastrointestinal tract is selected from mucocytis, colitis, Crohns disease, and inflammatory bowel disease.

39. Isolated TadE peptide.

40. Isolated TadE peptide of claim 39 comprising SEQUENCE ID NO: 30.

41. Isolated TadE peptide of claim 39 comprising SEQUENCE ID NO: 3.

42. An isolated nucleic acid encoding a TadE peptide of any of claims 39 to 41.

43. An isolated nucleic acid encoding a TadE peptide of claim 40 or 41.

44. An isolated nucleic acid according to claim 42 or 43 which is a cDNA molecule.

45. A bacterium expressing a Tad pilus, for use in a method of inducing proliferation of epithelial cells in a subject having epithelial cells damaged by disease, surgery, trauma, chemotherapy or radiotherapy.

46. A bacterium expressing a Tad pilus, for use of claim 45, in which the epithelial cells are gut epithelial cells.

47. A bacterium expressing a Tad pilus, for use in a method of development of gut epithelial lining in a premature infant

48. A bacterium expressing a Tad pilus, for use in a method of treating or preventing a disease or condition in a subject characterised by attenuated cellular growth of a target tissue.

49. A bacterium a Tad pilus, for use of any of claims 45 to 48, in which the bacterium is engineered for heterologous expression of the Tad pilus.

50. A bacterium expressing a Tad pilus, for use of any of claims 45 to 48, in which the Tad pilus comprises SEQUENCE ID NO: 30.

51. A bacterium expressing a Tad pilus, for use of any of claims 45 to 48, in which the Tad pilus comprises SEQUENCE ID NO: 3.

52. A bacterium expressing a Tad pilus, for use of any of claims 45 to 48, in which the bacterium is a strain of Bifidobacterium breve.

53. A bacterium expressing a Tad pilus, for use of any of claims 45 to 48, in which the bacterium is Bifidobacterium breve UCC2003.

54. A food or dietary supplement composition comprising an isolated TadE polyamino acid sequence.

55. A food or dietary supplement composition according to claim 54 in which the isolated TadE polyamino acid sequence comprises SEQUENCE ID NO: 30.

56. A food or dietary supplement composition according to claim 54 in which the isolated TadE polyamino acid sequence comprises SEQUENCE ID NO: 3.

57. A food or dietary supplement composition according to claim 54 in which the isolated TadE polyamino acid sequence comprises SEQUENCE ID NO: 1.

58. A food or dietary supplement composition comprising a bacterium according to any claims 45 to 53.


SEQUENCE LISTING (34 sequences)

SEQ ID NO: 1 - protein, 126 aa, Bifidobacterium breve
MNMRKGLSRWLARLPPVHNEGAATAEFAVVLPSVIAITGVLLTLTRVVAVSMDCQSAATASARELVVSDDESAAQQAAVNIVGSRVDVTVHREGSLVRVAVECPVLPGPLNVTPTRVHGEAVAARQ

SEQ ID NO: 2 - DNA, 381 bp, Bifidobacterium breve
atgaacatgagaaaaggtctcagcagatggctcgcccgtttgccgcctgtgcacaatgagggtgcggccacggccgagttcgcggtagtgctgcccagcgtcattgcaataacgggggtgttgctgacgcttacccgagtggttgcggtatccatggattgccaaagtgccgcaacggcatccgcgcgtgaattggtggtttctgatgatgaatccgcagcacaacaggctgccgtgaatattgtgggaagccgagtggatgtgactgtgcatcgggaaggcagcttggtgcgagtggcggtcgaatgccccgttttgccagggcctttgaatgtgactcctactcgggtgcatggtgaagctgtggctgcgcggcaataa

SEQ ID NO: 3 - protein, 66 aa, Bifidobacterium breve
SARELVVSDDESAAQQAAVNIVGSRVDVTVHREGSLVRVAVECPVLPGPLNVTPTRVHGEAVAARQ

SEQ ID NO: 4 - DNA, 201 bp, Bifidobacterium breve
tccgcgcgtgaattggtggtttctgatgatgaatccgcagcacaacaggctgccgtgaatattgtgggaagccgagtggatgtgactgtgcatcgggaaggcagcttggtgcgagtggcggtcgaatgccccgttttgccagggcctttgaatgtgactcctactcgggtgcatggtgaagctgtggctgcgcggcaataa

SEQ ID NO: 5 - DNA, 38 bp, Artificial Sequence
  tadZF
ctggaccccgggatgaatatcgacacatcctctcgcac

SEQ ID NO: 6 - DNA, 36 bp, Artificial Sequence
  tadCR
tgcgcatctagactacatcaggaaagaagcaattgc

SEQ ID NO: 7 - DNA, 36 bp, Artificial Sequence
  flpF
ctggaccccgggatggacacgcacattcccatcatc

SEQ ID NO: 8 - DNA, 33 bp, Artificial Sequence
  spkR
tgcgcaaagcttctagttaaagcgtccggtcac

SEQ ID NO: 9 - DNA, 32 bp, Artificial Sequence
  pNisF
ctggactctagactgacaatagaaacattaac

SEQ ID NO: 10 - DNA, 26 bp, Artificial Sequence
  tadVF
aattaaacatgtggtgcaggcgggtg

SEQ ID NO: 11 - DNA, 30 bp, Artificial Sequence
  tadVR
catcacggatcccaagacaaccccaacaac

SEQ ID NO: 12 - DNA, 30 bp, Artificial Sequence
  TadEHF
gacaagggatcctccgcgcgtgaattggtg

SEQ ID NO: 13 - DNA, 31 bp, Artificial Sequence
  TadEHR
agctctctagattattgccgcgcagccacag

SEQ ID NO: 14 - DNA, 32 bp, Artificial Sequence
  TadFHF
gacaagggatccgcacgttccctagctgacct

SEQ ID NO: 15 - DNA, 30 bp, Artificial Sequence
  TadFHR
agctctctagattactcactagcacctccc

SEQ ID NO: 16 - DNA, 24 bp, Artificial Sequence
  TadE soeA
gcaattgcttctttcctgatgtag

SEQ ID NO: 17 - DNA, 41 bp, Artificial Sequence
  TadE soeB
gagtaggagtcacattcaaaggaggcggcaaacgggcgagc

SEQ ID NO: 18 - DNA, 41 bp, Artificial Sequence
  TadE soeC
gctcgcccgtttgccgcctcctttgaatgtgactcctactc

SEQ ID NO: 19 - DNA, 21 bp, Artificial Sequence
  TadE soeD
cgtaccggtaatgtcgatgac

SEQ ID NO: 20 - DNA, 35 bp, Artificial Sequence
  TadE soeE
cctgcactgcaggcacggagtcaggagagcgcacg

SEQ ID NO: 21 - DNA, 33 bp, Artificial Sequence
  TadE soeF
gctagactgcagctcccttatcgcattccgccg

SEQ ID NO: 22 - DNA, 18 bp, Artificial Sequence
  TadF soeA
ctgagtacgcggtggtgc

SEQ ID NO: 23 - DNA, 36 bp, Artificial Sequence
  TadF soeB
cttatcgcattccgccggtgacatacgtggcataca

SEQ ID NO: 24 - DNA, 36 bp, Artificial Sequence
  TadF soeC
tgtatgccacgtatgtcaccggcggaatgcgataag

SEQ ID NO: 25 - DNA, 21 bp, Artificial Sequence
  TadF soeD
ctagttaaagcgtccggtcac

SEQ ID NO: 26 - DNA, 35 bp, Artificial Sequence
  TadF soeE
gctctactgcaggatgccattaagacgttgctgac

SEQ ID NO: 27 - DNA, 24 bp, Artificial Sequence
  TadF soeF
cttgacctgcaggaagtcacacgg

SEQ ID NO: 28 - DNA, 18 bp, Artificial Sequence
  pBSF
cgttacgttattagttat

SEQ ID NO: 29 - DNA, 19 bp, Artificial Sequence
  pBSF
gtaatacgttcgtgtcgcg

SEQ ID NO: 30 - protein, 10 aa, Bifidobacterium breve
VHGEAVAARQ

SEQ ID NO: 31 - protein, 18 aa, Bifidobacterium breve
PLNVTPTRVHGEAVAARQ

SEQ ID NO: 32 - protein, 29 aa, Bifidobacterium breve
RVAVECPVLPGPLNVTPTRVHGEAVAARQ

SEQ ID NO: 33 - protein, 12 aa, Bifidobacterium breve
TPTRVHGEAVAA

SEQ ID NO: 34 - protein, 11 aa, Bifidobacterium breve
TRVHGEAVAAR